(19) United States
(12) Patent Application Publication
(10) Pub. No.: US 2018/0140495 A1
(43) Pub. Date: May 24, 2018

(54) MULTIFUNCTION JOINT BRACE

(71) Applicant: Brett Behrendt, Harbor Springs, MI (US)

(72) Inventor: Brett Behrendt, Harbor Springs, MI (US)

(21) Appl. No.: 15/817,962

(22) Filed: Nov. 20, 2017

Related U.S. Application Data
(60) Provisional application No. 62/424,046, filed on Nov. 18, 2016.

(51) Int. Cl. A61H 1/00; A61F 5/02

(52) U.S. Cl. CPC A61H 1/008; A61H 2201/5082; A61H 2201/0207; A61F 5/02

(57) ABSTRACT

A multifunction joint brace that includes a belt that is wrapped about a user's waist, ankle, neck, wrist, hand or elbow and positions three electrically powered heating elements that are activated to apply low, medium, and high levels of heat to areas of muscle and tissue to relieve pain temporarily and increase blood flow to the affected area. The brace is in the central region. The brace also includes heating elements, a controller, a sensor for each respective heating element, a source of power, such as a rechargeable battery, and a logger for recording data from the sensors, each sensor measuring a different parameter or providing a power cut off if needed. The controller activates the heating elements for a preselected time and temperature and the sensors allow a therapist to review the data from the user with respect to effectiveness of the therapy.

### CROSS-REFERENCE TO RELATED APPLICATION

This application is a completion application which claims the priority benefit of co-pending U.S. Provisional Patent Application Ser. No. 62/424,046 filed Nov. 18, 2016, for “Multifunction Joint Brace,” the disclosure of which is hereby incorporated by reference in its entirety, including the drawing.

### BACKGROUND OF THE INVENTION

### 1. Field of the Invention

The present invention pertains to systems for treating or supporting human joints or a portion of the human body. Even more specifically the present invention is directed to multifunction orthopedic brace appliances used to wrap or support body parts, such as knees, elbows, hips, neck or other joints, the brace having multiple heating elements that may be selectively activated on enablement of respective setting levels and sensors that in real time control heating as well as feed a patient's biometric data to a computer.

### 2. Description of Related Art

Orthopedic braces are useful as preventative aids to prevent injuries to joints caused by motions or orientations of the joint that are outside the biomechanical limits of the joint. Orthopedic braces are also useful to promote proper healing of a joint following an injury to, or surgery on, the joint. Braces are also useful as a method to stabilize joints weakened as a result of disease or injury.

The following patent documents are illustrative of orthopedic brace appliances: U.S. Pat. No. 3,703,171; U.S. Pat. No. 7,507,215; U.S. Pat. No. 8,084,722; U.S. Pat. No. 8,425,529; U.S. Pat. No. 8,795,211; U.S. Pat. No. 9,072,898; US 2003/0052120; US 2005/0075593; 2006//0089582; US 2009/0012436; US 2013//0306614; US 2015/0025427; and US 2016/0213924. The above references are identified herein in recognition of a duty of disclosure of related subject matter, which may be relevant under 37 CFR 1.56, and are specifically incorporated, herein by reference as regards the conventional approaches and constructions taught therein.

For example, U.S. Pat. No. 3,703,171 discloses an athletic knee supporter and protective device suitable for both contact and noncontact sports. The device included a tapered elastic tubular element formed of two concentric tubes of elastic material for encompassing a user's leg above and below the knee. The brace included substantially planar stays, formed with obtuse angles, made of a rigid material and fitted within pockets formed in the tubular elements at both sides of the knee. It is believed that braces of this design provide mechanical support to a joint, but do not perform other functions.

Some brace designs have been enhanced to provide heat or other stimulus, in order to relieve discomfort or promote healing of an injured joint or other body part. For example, U.S. Pat. No. 7,022,093 discloses a self-contained heating and cooling orthopedic brace including a main body having straps to support the brace at various body areas, including the back, knee, elbow and shoulder. The brace further includes inner pockets having heating and cooling pads connected to a temperature controller that allows manual adjustment of the desired temperature.

In addition, U.S. Pat. No. 9,072,898 discloses an orthopedic brace system that provided electrical muscular stimulation (EMS) therapy to an affected joint or other body portion. The system includes a sensor in contact with human tissues of a patient and is configured to obtain a power dissipation reading of the human tissues. The system also includes a control unit in communication with the brace to instruct the sensor to (a) apply a sense pulse to the human tissues, (b) measure power dissipation of the sense pulse, (c) adjust a stimulation pulse based on the measured power dissipation, and (d) apply the stimulation pulse to the human tissues based on the power dissipation.

The foregoing orthopedic brace designs have limited functionality and are unable to collect and process biometric information that can be used to monitor use of the device or customize the function of the brace to the needs of an individual user.

Further, the brace designs in the patent documents referenced above do not disclose a multifunction brace system that includes a heating feature of heating elements provided within one or more respective channel strands of a brace that can be selectively activated based on enablement of one of three respective setting levels.

Further the references do not specifically disclose the aforementioned multifunction brace system that, in addition, provides a combination of data from a plurality of sensors along with information regarding a temperature and time of duration of heat being applied at respective setting levels that may be tracked, logged, stored, and/or communicated to external devices (e.g., wirelessly) to provide real time data with respect to an effectiveness therapy.

The present invention is directed to this multifunction brace system.

### SUMMARY OF THE INVENTION

The present invention is a multifunction brace, comprising: a belt for placement about or on a user body, said belt having, (a) an inner surface adapted to be wrapped about the body and (b) a central region adapted to be positioned about a selected area of the body to which heat therapy is to be applied. At least two heating elements are embedded at predetermined juxtaposed locations in the central region. The brace also, includes a module for selectively activating the heating elements based on enablement of the respective heating elements reaching one of two respective setting levels. A control unit is positioned in the central region of said belt, the control unit operably associated with the module, and a power source for powering the control unit. Preferably, three heating elements are provided, each connected to a respective sensor, and a data logger is provided.

This configuration will allow a patient(s) to be mobile with the support of the brace to support the targeted body part to stay in its normal range of motion and also use deep dry heat applied to the body in a targeted area at three different temperature levels and specific durations of heating therapy to reduce pain and assist in the healing process.

Preferably, the heating element is made out of a flexible woven mesh that has its heat element strands made out resistance wire, threads and substrates within the mesh. The heating element strands are woven through the mesh by three separate channels.

According to the invention, the heating elements provide three different selectively controlled levels of heat to the patient. The heating element provides dry heat into the deep tissues that focus relief in on muscle “knots” or trigger points, and most types of cramping/spasms. Where the cause of the pain is result of muscles becoming shorter or restricted, the targeted heat will limber up the muscles and help allow them to be stretched with therapy.

Preferably, the sensors include a triaxial sensor, a gyro sensor, and a thermistor. The tri-axial sensor is a sensor which measures brace movement in three axes—horizontal, vertical, axial. This data provides feedback on how the brace is holding the body part in place. The sensors provide a simultaneous measurement of alignment in 3 perpendicular plains. These measurements are known in the industry as “deflections”. The deflections provide the range of motion of the body part.

The gyro sensors provide a data log on movement and intensity levels. Gyro sensor data combined simultaneously, with the heating element temperature and the tri-axis sensor along with the amount of movement will chart combined results over time. This log can enable the amount of activity that the patient is doing every day to be charted. In some embodiments of the multifunction brace, a healthcare professional may be able to determine if the patient is doing the required exercise, or is the patient overdoing the recommended amount, and what the patient's progress in between the therapy sessions or doctor visits.

The controller or control unit and thermistor may also monitor the heat levels for safety and will shut down if the temperature hits a maximum surface temperature.

The controller or control unit will, preferably, comprise an open system to share the stored data with use of Blue Tooth/cellular communication. The information can be paired with PC's and Smartphones. Alternatively, the data can be rendered transferrable with a USB drive.

The controller, along with an on-board data logger, provides continuous sensors data input. The data input continuously logs the simultaneous feedback of the battery along with the triaxial, gyro, and thermistor sensors.

The controller and data logger can provide a log of simultaneous measurement of what angles of movement are achieved along with the temperature level. This provides real time data on the effectiveness of the heating element setting and what the patient's range of movement is with and without applied heat. Such data may help the health care professional to adjust therapy to match the improvement of the body part. This also shows if the patient is hyperextending the body part or bowing out to the sides and out of alignment. The gyro sensors can provide a data log on movement and intensity levels. The gyro sensor data combined, simultaneously, with the heating element temperature with the triaxial sensor along with the amount of movement can chart combined results over time. This log may enable the amount of activity that the patient is doing every day to be charted.

In some embodiments of the multifunction brace, a healthcare professional may be able to determine if the patient is doing the required exercise or is the patient overdoing the recommended amount and the patents progress in between therapy sessions and/or doctor visits. The controller will use an open system to share the stored data with use of Blue Tooth/cellular communication. Information can be paired with PC's, Apple and Smartphones. Alternatively, the data can be transferred with a USB drive.

The low setting level of heating only activates the first channel strands. This setting is for low heat and is woven through the mesh in a wide path. This distance, with only the first channel activated, will heat the entire surface area of the heating element panel to a temperature of about 105° F. for a duration of about 30 minutes. The about 105 degrees temperature is maintained at a constant level with the use of the thermistor along with the controller. The controller uses the data logger to record the surface temperature and the deflection angles as well as managing the power output of the battery. The controller and thermistor also monitor the heat levels for safety and will shut down if the temperature hits a maximum surface temperature of about 175° F. This will only allow the heating element to operate in a safe temperature range of between 0° to about 175° F.

The medium setting level activates the first channel along with the second channel heating element strands. The second channel strands form a path that is half way between the paths of the level one strands. The additional heat generated by tuning channel one along with level two will heat the entire surface area of the heating element panel to a temperature of about 145° F. for a duration of about 20 minutes. The about 145° F. temperature is maintained at a constant level with the use of the Thermistor along with the controller. The controller also uses the data logger to record the surface temperature and the deflection angles and the controller manages the power output of the battery. The controller and the thermistor will also monitor the heat levels for safety and will shut down if the temperature hits a maximum surface temperature of about 175° F. This will only allow the heating element to operate in a safe temperature range of about (0°-175° F.).

The advantages of applying constant heat over the entire surface area at fixed temperature levels and specific time settings advantageously accomplishes deep tissue heat to the entire area of injury and penetrates down to where the muscles are at the bone.

The flexible open mesh type material helps with air flow through the fabric assists in preventing overheating of a specific body part and helps against (skin irritation and sores). The open mesh material is made out of a washable, flexible, non-allergenic material.

For a better understanding of the present invention, reference is made to the accompanying drawing and detailed description. In the drawing, like reference numerals refer to like parts through the several views, in which:

### BRIEF DESCRIPTION OF THE DRAWINGS

FIGS. 1A, 1B and 1C show a multifunction joint brace according to this invention being wrapped around the waist and or hip area of a user with heating elements of the brace disposed at the back and proximate the spine;

FIG. 2 is an enlarged view showing an inner surface of the multifunction joint brace and a support pad centrally of the brace having arranged thereon aspects of the brace including a heating arrangement in a woven mesh element, a control, sensors, a module for selectively activating the heating elements, a power source, and a data logger; and

FIG. 3 is an enlarged view showing detail of the support pad of FIG. 2 and elements thereof.

### DETAILED DESCRIPTION OF THE INVENTION

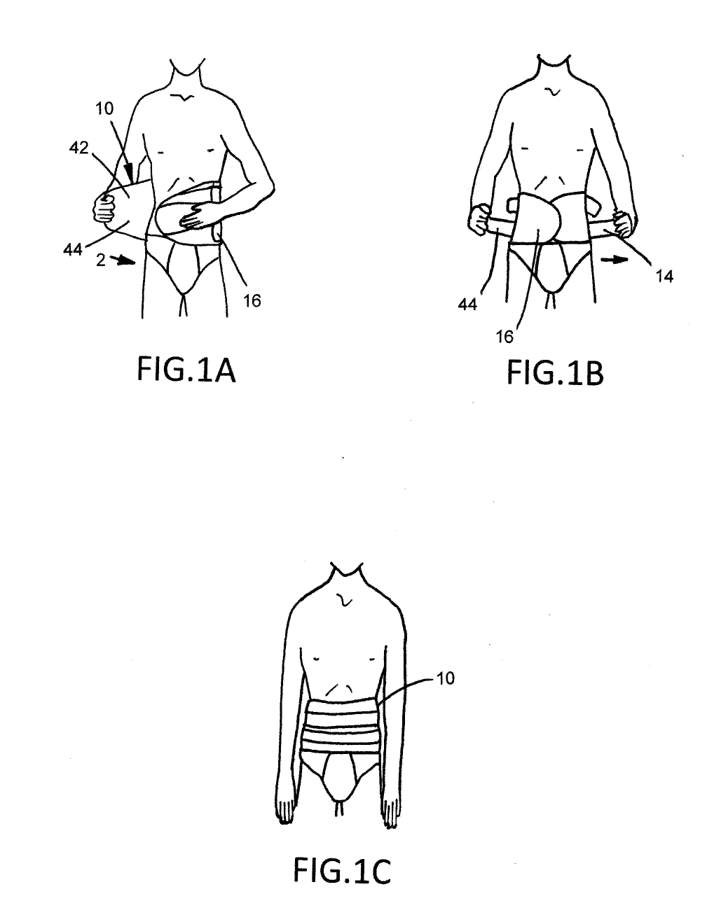

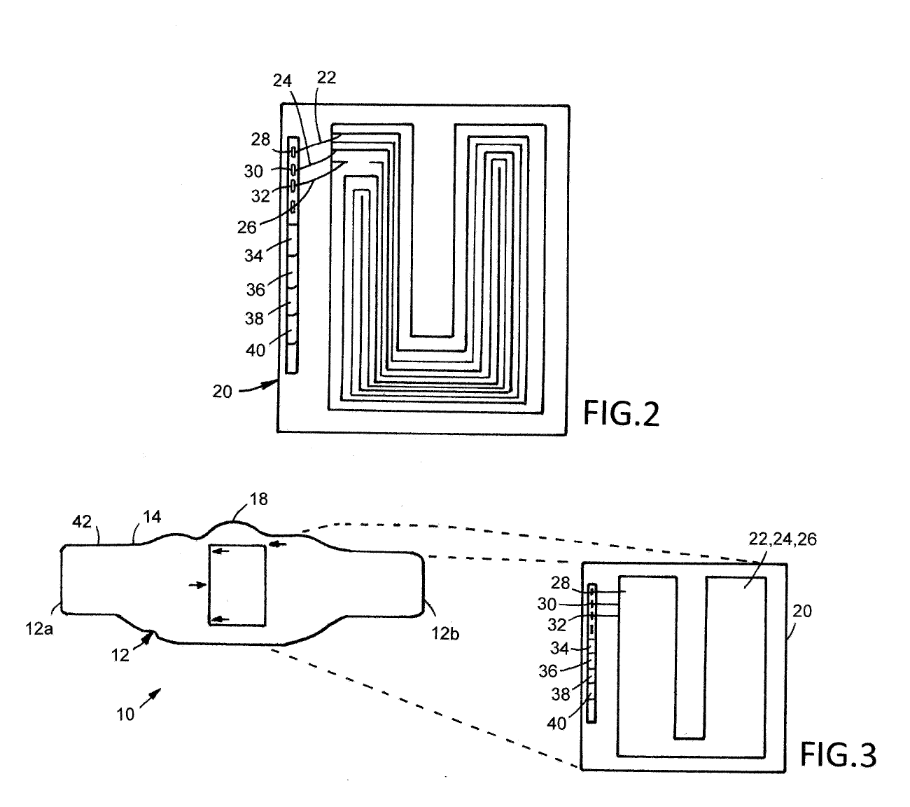

Now, and in accordance with the present invention, and with reference to FIGS. 1-3 of the drawing, there is provided, a wrap-around style multifunction joint brace 10 in the form of a belt 12 with an inner lining or surface 14 and a closure 40 at the opposite ends 12a and 12b of the brace. The belt has a central region 18. A support pad 20 is arranged or mounted to a central region 18 is a support pad 20, which includes a rechargeable battery or power source 38, battery controlled heating elements 22, 24 and 26, a module 34 for selectively activating the heating elements based on enablement of the respective heating elements reaching one of two respective setting level, a control unit or controller 36, the control unit being operably associated with the module and powered by the battery, a data logger 44, and sensors 28, 30, and 32 electrically connected to the controller 36.

The multiple heating elements are disposed in a woven mesh and may be selectively activated on enablement of respective setting levels. The sensors in real time control heating and feed a patient's biometric data to a computer.

The brace 10 may be used to provide support to a joint or body part which has been diagnosed with a condition, injury and or is recovering from a surgical repair to any body parts, such as knees, elbows, hips, neck or other joints. The brace provides strength to the body part during the healing process. The brace should also allow the range of motion not to exceed the normal healthy range of movement for the particular joint or body part involved, and maintain normal alignment of the body part so as not to strain or damage the joint to associated structures, such as ligaments.

As shown in FIGS. 1A-1C, the brace 10 is shown as being fitted about the waist or hip area of a user with the inner surface 14 of the belt contacting the user. The support pad 20 at the central region 18 is positioned against the user's spine region. The closure 40 includes the ends 12a and 12b and straps 44 at the opposite ends. Once the ends 12a and 12b of the brace are joined, as in FIG. 1B, the closure straps 42 are releasably tightened and the brace is firmly in place about the user.

Preferably, the brace 10 comprises an outer brace (or wrap) and an inner brace. The outer brace can be one of any conventional designs and may be made from open mesh Nylon hypoallergenic material. The brace is dimensioned to be of a wraparound design, completely enclosing the joint or body part to be supported. The outer brace is held in place by a fastening mechanism, or closure 40, such as an adjustable strap, buckles, “loop and hook” fasteners (such as Velcro®), or any similar fastening mechanism. The wrap design allows easy placement and removal of the wrap by patients of all ages.

The outer brace, preferably, includes means for adjusting the amount of lateral pressure applied to the joint or affected body part by selecting the appropriate amount of tension using the fastening mechanism. For example, the lateral pressure applied to a knee race can be adjusted by tightening or loosening an adjustable strap(s) 44 through the use of a loop and hook fastener.

The outer brace is preferably dimensioned so that it can be fitted properly to users with different proportions and to be relatively easy and simple to use, maintain, and store.

The inner brace provides internal support to the joint or affected body portion. The inner brace preferably has an element providing internal reinforcement, such as a stay or rigid skeleton. The internal reinforcement element is useful in providing additional support and alleviating pain. For example, and internal reinforcement element in a knee brace if properly adjusted helps to alleviate knee pain by reducing the weight on the users medial or lateral tendons

The inner brace preferably contains a support mechanism to control the range of motion of the joint or affected body part. A controlled range of motion can prevent the user from bending the joint in a manner that exceeds the normal healthy range of movement, placing strain on the joint and potentially aggravating the injury. For example, a knee brace may contain a mechanism for controlling range of motion to prevent the hyperextension of the knee.

A preferred material for the inner brace is a neoprene free open mesh-type of nylon material. The open mesh-type of nylon material also allows air to circulate into an out of the brace, reduces the weight of the brace, and allows the brace unit to be washed and dried without damaging brace material. Preferably, the brace material should be non-allergenic to a wide range of potential users. A person skilled in the art will appreciate that braces according to the parameters described herein can be made out of any suitable material, including plastics, nylon fabrics, and is a hypo-allergenic material that will not cause irritation when worn directly on the skin.

The brace further includes internal battery-powered heating elements 22, 24, and 26 and a control module 34 that allows the patient to be mobile with the support of the brace and also have heat applied to the body area to reduce pain and assist in the healing process. The module 34 selectively activates the heating elements based on enablement of the respective heating elements reaching one of two respective setting level. The heating elements provide heat into the deep tissues that will focus relief in on muscle “knots” or trigger points, and most types of cramping/spasm. When the cause of the pain is a result of the muscles becoming shorter or restricted. The targeted heat will limber up the muscles and help allow them to be stretched with therapy

A power source or system 38 comprises a suitable battery power device, such as a portable lithium ion battery powered unit, or other suitable portable battery power source. Due to the energy requirements to power the brace heating unit, the batteries must have a high capacity. The battery power system may be rechargeable using an external power source, such as an A/C wall power charger, USB power source, or “cigarette-type” port power charger.

Advantageously, the brace is portable, allowing the user to go through their daily activities without being restricted to an A/C power cord or connection to another fixed source of electrical power.

Preferably, the sensors 28, 30 and 32 comprise a triaxial sensor, a gyro sensor, and a thermistor.

The triaxial sensor 28 measures brace movement in three axes—horizontal, vertical, axial. This data provides feedback on how the brace is holding the body part in place and are known in the industry as “deflections”. The measurement of deflections will provide simultaneous data concerning the range of motion of the body part or affected joint, including extent and angles of motion. This data concerning the movement of the brace in use provides a healthcare professional with data that may be used to evaluate the performance of the brace and the effectiveness of the therapy, such as whether the brace is holding the body part in place during therapy or other motion. The triaxial sensor also may monitor if the brace is correctly fitted and providing the correct alignment for the effected joint.

The controller 36 may provide a simultaneous measurement what angles of movement are achieved along with the selected temperature level. The combination of data from the triaxial sensor and heat and time of duration information can provide real time data on the effectiveness of the heating element setting and feedback data to permit a healthcare professional to evaluate the effectiveness of therapy. For example, the data may indicate a patient's range of movement with and without applied heat. This data will help the health care professional to adjust any therapy to match the improvement of the body part. In addition, the data can reveal whether a patient is performing a prescribed therapy incorrectly, such as by hyper extending a body part or bowing out of alignment.

The gyro sensor 30 simultaneously and continuously collects data concerning the patient movement of the joint of interest. The gyro sensor may be electronically connected to the controller 36, providing yet another source of information to evaluate the movement of the affected joint and allow a health practitioner to determine whether the therapy is effective. A brace may have one or more gyro sensors placed to collect data of interest. The gyro sensors may log movement and intensity levels by providing location and movement information to the controller. Data from the gyro sensors may be combined with information concerning the heating element temperature and motion information collected by the triaxial sensors to provide a comprehensive profile of the patient's therapy over time, including temperature, duration, extent of movement, presence of abnormal or unwanted movement, and a record of the pattern of the patient's therapy, including time, date, and duration. This information can be collected and stored by the controller using a electrically connected data storage device such as the data logger 40.

The thermistor 32 is a type of resistor whose resistance is dependent on temperature, as a safety feature. The thermistor will cause the heating function of the brace to shut down if the temperature at the point of contact with the user's skin reaches a predetermined maximum surface temperature. The maximum surface temperature may be pre-established to a temperature of about 100° to about 175° F., preferably a temperature of about 175° F.

In addition to the each one or more tri-axial, gyro and thermistor sensors, additional each of one or more sensors, such as torsiometers, goniometer-torsiometer, as well as, tapered whisker sensors for estimating radial distance may be used. Also, microelectromechanical sensors (MEMS) can be incorporated into the brace. These additional devices, optimally, would be in communication with the controller and/or data logger.

The high setting level activate the first channel along with the second channel heating element strands and activating the third channel strands. The third channel lie in a path that is half way between the path of the level one strands and the strands of level two strands. The additional heat generated by tuning channel one along with channel two and channel three will heat the entire surface area of the heating element panel to a temperature of about 165° F. for a duration of about 20 minutes. The about 165° F. temperature is maintained at a constant level with the use of the thermistor along with the controller 36. The controller 36 also uses the data logger 40 to record the surface temperature and the deflection angles and the controller manages the power output of the battery 38. The controller and thermistor also monitor the heat levels for safety and will shut down if the temperature hits a maximum surface temperature of about 175° F. This will allow the heating element to operate in a safe temperature range of between about 0° to 175° F.

The brace may have multiple preset dry heating element levels, and each preset level may include a predetermined treatment duration. In one embodiment the brace 10 includes three intensity levels to allow the medical professional the ability to prescribe the intensity level to match the progression of the patient's healing. Other braces may have more or fewer intensity levels, depending on their intended use.

The control unit 36 of the brace is connected to the heating element(s) to allow patients to actuate the heating elements prior to physical therapy sessions. This will allow the body part to be warmed up prior to therapy and allow the therapist to get more results for each session. A person skilled in the art will recognize that without heat a physical therapist may be required to spend the first 45 minutes of a physical therapy session to allow the patient's body part warmed up and the last 15 minutes is the actual therapy during an hour-long session.

According to the present invention, the brace includes a first intensity level (“Level 1 Low”) that permits the heating elements 22, 24, and 26 in the brace in contact with the patient's skin in a targeted area to reach a surface temperature not to exceed about 105° F. for time duration of about 30 minutes. This will be a continuous dry heat over the entire targeted surface area. This Level 1 Low setting may be particularly appropriate for patients in one or more of the following scenarios:
i) Low and steady for recent trauma/surgery
ii) Also to be used as a warm up setting for mornings and pre therapy sessions
iii) Help limber up the body part to increase range of motion
iv) Reduce pain
v) The concentration of heat will speed up healing to the body part
vi) Used after therapy or exercise to reduce the pain after exertion

The brace also, preferably, includes a second intensity level (“Level 2 Medium”) that permits the heating elements in the brace in contact with the patient's skin to reach a surface temperature not to exceed 145° F. for time duration of about 20 minutes. This will be a continuous dry heat over the entire targeted surface area. This Level 2 Medium setting may be particularly appropriate for patients in one or more of the following scenarios:
i) Reduce chronic pain during healing
ii) The concentration of heat will speed up healing to the body part
iii) Help calm muscle spasms during healing

The brace, also, preferably includes a third intensity level (“Level 3 High”) that permits the heating elements in the brace in contact with the patient's skin to reach a surface temperature not to exceed 175° F. for time duration of about 20 minutes. This will be a continuous dry heat over the entire targeted surface area. This Level 3 High setting may be particularly appropriate for patients in one or more of the following scenarios:
i) For deep tissue warming
ii) Deep penetration to wear the muscle meets the bones
iii) Reduce chronic pain during healing
iv) Help calm extreme muscle spasms during healing

In some embodiments, the brace may be adapted to include additional pre-determined heat and time settings depending on the needs of the patient. The settings may be selected by the patient or by a health care provider. As noted herein, sensors in the brace record the patient's use of each setting. The brace may include a controller to control the heating levels and monitor information from sensors which will be stored on a data logger which will provide critical data back to the health care professional This will log continuous/simultaneous deflection angles of the joints along with the surface skin temperature and the amount of activity levels which the specific joint has been subjected/used.

The electronic control unit 36 is electrically connected to the heating unit module 34 and a timer (not shown) to control the multiple functions of the brace. The functions which the controller may manage include:
v) All on-board data logging and continuous sensors data input
vi) the thermistor (the controller will shut down the heating element if the surface temperature reaches about 175° F.).
vii) the feedback from a triaxial sensor, such as a triaxial accelerometer.

The triaxial sensor 28 provides simultaneous angular measurements in three perpendicular axes: horizontal, vertical, and axial. The triaxial sensor collects data concerning the movement of the brace in use, providing a healthcare professional with data that may be used to evaluate the performance of the brace and the effectiveness of the therapy, such as whether the brace is holding the body part in place during therapy or other motion. The triaxial sensor may provide the simultaneous measurement of alignment in 3 perpendicular planes. The triaxial sensor also may monitor if the brace is correctly fitted and providing the correct alignment for the effected joint.

The triaxial sensor 28 also may measure deflections of the brace and indicate the motions of the affected joint. The deflections will provide simultaneous data concerning the range of motion of the body part, including extent and angles of motion. The controller 36 may provide a simultaneous measurement what angles of movement are achieved along with the selected temperature level. The combination of data from the triaxial sensor and heat and time of duration information may provide real time data on the effectiveness of the heating element setting and feedback data to permit a healthcare professional to evaluate the effectiveness of therapy. For example, the data may indicate a patient's range of movement with and without applied heat. This data will help the health care professions to adjust therapy to match the improvement of the body part. In addition, the data will reveal whether a patient is performing a prescribed therapy incorrectly, such as by hyper extending a body part or bowing out of alignment.

The gyro sensor 30 simultaneously and continuously collects data concerning the patient movement of the joint of interest. The gyro sensors may be electronically connected to the controller 36, providing yet another source of information to evaluate the movement of the affected joint and allow health practitioner to determine whether the therapy is effective. A brace may have one or more gyro sensors placed to collect data of interest. The gyro sensors may log movement and intensity levels by providing location and movement information to the controller. Data from the gyro sensors may be combined with information concerning the heating element temperature and motion information collected by the triaxial sensors to provide a comprehensive profile of the patient's therapy over time, including temperature, duration, extent of movement, presence of abnormal or unwanted movement, and a record of the pattern of the patient's therapy, including time, date, and duration. This information can be collected and stored by the controller using a electrically connected data storage device.

The combined information over time provide a health practitioner with a valuable source of information to assess a patient's therapy and, if appropriate, make adjustments or provide detailed instructions to improve the therapeutic results. For example, the data may include:
i) A log of the amount of activity that the patient is doing every day.
ii) Whether the person is actually performing the prescribed exercise or therapy.
iii) Whether the patient is exceeding the recommended amount of exercise or therapy.
   This will provide real time information which can be charted of the patient's progress in between the therapy sessions or doctor visits.

In addition, the controller may be connected to a storage or communication device using a wireless network interface, and may be adapted to use an open system to share the stored data with use of Blue tooth/cellular communication. In this way, the information collected concerning the therapy can be shared with a remote computing dev ice, such as a personal computer, tablet computer, smart phone or other mobile device. Alternatively, the brace may be configured to allow the data can be transferred with a USB drive or other removable storage device.

In some embodiments of the multifunction brace, one or more of the sensing devices may be located in a biometric surface strip (a “bio strip”) attached to the brace. The bio strip (not shown) is conventional and may include:
i) a gyro sensor to log patient movement;
ii) a triaxial sensor to log patient deflections; and
iii) a thermistor to monitor surface temp and also work as a power kill switch to shut down all power if the surface temperature exceeds 175 degrees Fahrenheit.

In addition, the multifunction joint brace 10 preferably includes an on-board data logger 40 to store continuous data input from one or more sensors, including those located on a bio strip. The data logger (or data recorder) is an electronic device that records data over time or in relation to location either with a built-in instrument or sensor or via external instruments and sensors. Increasingly, but not entirely, they are based on a digital processor (or computer).

The data logger is preferably a small, battery powered, portable, and equipped with a microprocessor, internal memory for data storage, and sensors. The data logger may interface with a personal computer or other external smart device, and use software to activate the data logger and view and analyze the collected data. Alternatively, the data logger may have a local interface device (keypad, LCD) and can be used as a stand-alone device.

Depending on the intended application and use, the data logger vary between general purpose types for a range of measurement applications to very specific devices for measuring in one environment or application type only. It is common for general purpose types to be programmable; however, many remain as static machines with only a limited number or no changeable parameters. Electronic data loggers have replaced chart recorders in many applications.

One of the benefits of including a data logger in the multifunction brace 10 is the ability to automatically collect data on a 24-hour basis. Upon activation, the data logger is deployed and left unattended to measure and record information for the duration of the monitoring period. This allows for a comprehensive, accurate picture of the environmental conditions being monitored, such as air temperature and relative humidity.

The data logger and the sensors, including those located on a Bio Strip, may provide added functionality to the multifunction brace, and provide a healthcare professional with data that may be used to evaluate the performance of the brace and the effectiveness of therapy. Among other things, this could include providing data feedback and monitoring the activity levels, and providing key physiological data during rehabilitation.

The controller 36 may employ any suitable software adapted to direct the essential functions of the multifunction brace. The controller may use open-source software (OSS) to enable the controller and multifunction brace sensors to communicate with external devices using compatible OSS programs, including in a wireless data connection, such as a Bluetooth® communication protocol.

Alternatively, data from the brace 10 can be transferred with a USB drive. Many types of consumer electronics support USB interfaces. These types of equipment are most commonly used for computer networking:
USB network adapters
USB broadband and cellular modems for Internet access
USB printers to be shared on a home network
For computer-to-computer file transfers without a network, USB drives are also sometimes used to copy files between devices.

From the above, it is to be appreciated that defined herein is a new and unique multifunction joint brace appliance used to wrap or support body parts, such as knees, elbows, hips, neck or other joints, the brace having multiple heating elements that may be selectively activated on enablement of respective setting levels and sensors that in real time control heating and feed a patient's biometric data to a computer.

### LIST OF REFERENCE NUMBERS

10 multifunction joint brace

12 belt

12a opposite end/12

12b opposite end/12

14 inner surface/12

16 outer surface/12

18 central region/12

20 support plate

22 heating element (a wire)

24 heating element (a wire)

26 heating element (a wire)

28 (triaxial) sensor

30 (gyro) sensor

32 (thermistor) sensor

34 module

36 controller/control unit

38 power source (battery)

40 data logger

42 closure arrangement/12a, 12b

44 tightening straps/12a, 12b

1. A multifunction joint brace, comprising
a belt for placement about a user body, said belt having an inner surface adapted to be wrapped about the body, the inner surface including a central region adapted to be positioned about and in engagement with a selected area of the body to which heat therapy is to be applied,
at least two heating elements embedded in the belt and at predetermined locations juxtaposed within the central region,
a module for selectively activating the heating elements based on enablement of the respective heating elements reaching one of two respective setting levels,
a control unit operably associated with the module, and
a power source for powering the control unit and said module whereby to activate the heating elements.

2. The multifunction brace according to claim 1, further wherein the brace comprises three heating elements, each of said heating element being activatable by said module to a different temperature level.

3. The multifunction brace according to claim 2, further including a sensor for each heating element, each sensor operably connected to a respective heating element for measuring and monitoring the heating time and the temperature in the respective heating element and operably connected to said module, and wherein the control unit is operably connected to said module for sending a signal, based on the data from said sensors, to said module for adjusting the power and activation time of said respective heating element.

4. The multifunction brace according to claim 3, further wherein one sensor is a thermistor, said thermistor terminating activation of the respective heating element based on a reading of a preselected time or an achieved temperature.

5. The multifunction brace according to claim 3, further including a data logger, and means for transmitting the time and temperature data recorded by the sensors to the data logger.

6. The multifunction brace according to claim 1, further wherein each said heating element is adapted to generate a different heat in the central region.

7. The multifunction brace according to claim 6, further comprising a first, second and third sensor and a first, second and third heating element, each sensor associated with a respective first, second and third heating element, and each of said heating elements being activatable to a preselected temperature, the temperature of the second heating element being activatable to a temperature greater than that of said first heating element and to a temperature lower than that of said third heating element.

8. The multifunction brace according to claim 7, further wherein said first sensor comprises a triaxial appliance that measures and provides data in the form of brace movement in each of the three coordinate x, y, and z-axes, said second sensor is a gyro appliance which measures rotation, and said third sensor comprises the thermistor, the thermistor places a limit on the temperatures reached by the heating elements and provides power cut off.

9. The multifunction brace according to claim 1, further wherein the power source is reusable and rechargeable.

10. The multifunction brace according to claim 1, further wherein the central region is positioned adjacent a user's back region when the brace is placed in use.

11. The multifunction brace according to claim 10, further wherein the central region is alignable with a portion of a user's spine.

12. The multifunction brace according to claim 1, further wherein the brace is longitudinally elongate and includes opposite ends, and the central region is disposed between the opposite ends, and further wherein said brace includes a closure mechanism for positioning and closing the belt on the user body, the closure mechanism operating to engage and hold the opposite ends together and in removably locked relation.

13. The multifunction brace according to claim 12, further wherein the brace includes a pair of tightening straps, the straps being on one and the other end of the brace and adapted to further position and tighten the brace about the user's body,

14. The multifunction brace according to claim 1, further wherein the inner surface of the belt, including the heating elements, comprises an open-mesh material that is neoprene free, and which allows air to circulate into and out of the brace, while inhibiting retention of moisture.

15. The multifunction brace according to claim 12, further wherein said control unit is remote to said belt and said module for activating said heating elements is wirelessly connected to said remote controller, said remote controller receiving real time data from said heating elements for adjusting the temperatures in the respective heating elements.

16. The multifunction brace according to claim 12, further wherein said control unit is positioned in the central region of said belt.

17. The multifunction brace according to claim 7, further wherein the heating elements are activatable to a first heat level not to exceed about 105° F., for a time duration of about 30 minutes, a second heat level not to exceed about 145° F. for a duration of about 20 minutes, and a third heat level of about 165° Fahrenheit for a time duration of about 20 minutes.

18. The multifunction brace according to claim 8, further wherein the thermistor allows the heating element associated therewith to reach a maximum temperature that does not exceed about 175° Ft, the thermistor sending a signal to the module to shut down if the maximum temperature is reached.

19. The multifunction brace according to claim 1, further wherein said belt comprises an inner brace connected to an outer brace, the inner brace engaging the user's body, and further wherein said inner brace includes an internal reinforcement element to provide support and alleviate pain by controlling the range of motion of the affected body part.
